(19) United States
(12) Patent Application Publication
(10) Pub. No.: US 2024/0339186 A1
(43) Pub. Date: Oct. 10, 2024

(54) MEDICAL SUPPORT SYSTEM, REPORT CREATION SUPPORT METHOD, AND INFORMATION PROCESSING APPARATUS

(71) Applicant: OLYMPUS MEDICAL SYSTEMS CORP., Tokyo (JP)

(72) Inventors: Haruhiko SAKAYORI, Tokyo (JP); Megumi NAGASAWA, Tokyo (JP); Emi MASUDA, Tokyo (JP)

(73) Assignee: OLYMPUS MEDICAL SYSTEMS CORP., Tokyo (JP)

(21) Appl. No.: 18/746,796

(22) Filed: Jun. 18, 2024

Related U.S. Application Data
(63) Continuation of application No. PCT/JP2022/001461, filed on Jan. 17, 2022.

(51) Int. Cl. G16H 15/00; G16H 10/60; G16H 30/40

(52) U.S. Cl. CPC G16H 15/00; G16H 10/60; G16H 30/40

(57) ABSTRACT

An image acquisition unit acquires a plurality of endoscopic images captured in an endoscopic examination. An additional information acquisition unit acquires respective pieces of additional information of the plurality of endoscopic images. An image selection unit selects a predetermined amount of report attachment images from among the plurality of endoscopic images based on priority order set for the pieces of additional information when no endoscopic image including a lesion that is to be attached to a report is detected by the image analysis device in the endoscopic examination.

### CROSS-REFERENCE TO RELATED APPLICATIONS

This application is based upon and claims the benefit of priority from the International Application No. PCT/JP2022/001461, filed on Jan. 17, 2022, the entire contents of which are incorporated.

### BACKGROUND

### 1. Technical Field

The present disclosure relates to a medical support system, an information processing apparatus, and a report creation support method for supporting report creation.

### 2. Description of the Related Art

In endoscopic examination, a doctor observes endoscopic images displayed on a display device and, upon finding a lesion, operates an endoscope release switch to capture (save) endoscopic images of the lesion. After the examination is completed, the doctor creates a report by inputting examination results on a report input screen and selecting endoscopic images to be attached to the report from the multiple captured endoscopic images. JP 2017-86274A discloses a report input screen that displays a list of the multiple captured endoscopic images as candidate images for attachment. JP 2020-81332A discloses a system that recognizes a treatment or a lesion on a subject from an endoscopic image and selects an endoscopic image to be used for a report.

In recent years, research and development of computer-aided diagnosis (CAD) systems have been underway. When creating an examination report, it is expected that the burden of the doctor's report creation work can be reduced through automatic selection of images in which lesions are detected by a CAD system as images to be attached to the report. In an examination in which the CAD system does not detect a lesion, there is no image in which the CAD system detects a lesion; however, even in such a case, it is preferable to be able to efficiently support the report creation work.

### SUMMARY

The present disclosure has been made in view of the aforementioned problems and a general purpose thereof is to provide a technology for supporting report creation work when endoscopic images including lesions to be attached to a report are not detected by a computer such as a CAD system.

A medical support system according to one embodiment of the present disclosure includes: one or more processors including hardware, wherein the one or more processors are configured to: acquire a plurality of endoscopic images captured in an endoscopic examination; acquire respective pieces of additional information of the plurality of endoscopic images; and select a predetermined amount of report attachment images from among the plurality of endoscopic images based on priority order set for the pieces of additional information when no endoscopic image including a lesion that is to be attached to a report is detected by a computer in the endoscopic examination.

A report creation support method according to another embodiment of the present disclosure includes: acquiring a plurality of endoscopic images captured in an endoscopic examination; acquiring respective pieces of additional information of the plurality of endoscopic images; and selecting a predetermined amount of report attachment images from among the plurality of endoscopic images based on priority order set for the pieces of additional information when no endoscopic image including a lesion that is to be attached to a report is detected by a computer in the endoscopic examination.

An information processing apparatus according to yet another embodiment of the present disclosure includes: one or more processors including hardware, wherein the one or more processors are configured to: acquire a plurality of endoscopic images captured in an endoscopic examination; acquire respective pieces of additional information of the plurality of endoscopic images; and select a predetermined amount of report attachment images from among the plurality of endoscopic images based on priority order set for the pieces of additional information when no endoscopic image including a lesion that is to be attached to a report is detected by a computer in the endoscopic examination.

Optional combinations of the aforementioned constituting elements and implementations of the present disclosure in the form of methods, apparatuses, systems, recording mediums, and computer programs may also be practiced as additional modes of the present disclosure.

### BRIEF DESCRIPTION OF THE DRAWINGS

Embodiments will now be described, by way of example only, with reference to the accompanying drawings which are meant to be exemplary, not limiting, and wherein like elements are numbered alike in several Figures, in which:

FIG. 1 is a diagram showing the configuration of a medical support system according to an embodiment;

FIG. 2 is a diagram showing functional blocks of a server device;

FIG. 3 is a diagram showing examples of additional information linked to endoscopic images;

FIG. 4 is a diagram showing functional blocks of an information processing apparatus;

FIGS. 5A to 5C are diagrams showing examples of priority order set for organs;

FIG. 6 is a diagram showing examples of priority order set for sites of organs;

FIG. 7 is a diagram showing priority order specified based on organ priority order and site priority order;

FIGS. 8A to 8C are diagrams showing examples of priority order set for the order of imaging;

FIG. 9 is a diagram showing an example of a report that is automatically created;

FIG. 10 is a diagram showing an exemplary variation of the priority order set for combinations of organs and sites; and

FIG. 11 is a diagram showing an exemplary variation of the priority order set for combinations of organs and sites.

### DETAILED DESCRIPTION

The disclosure will now be described by reference to the preferred embodiments. This does not intend to limit the scope of the present disclosure, but to exemplify the disclosure.

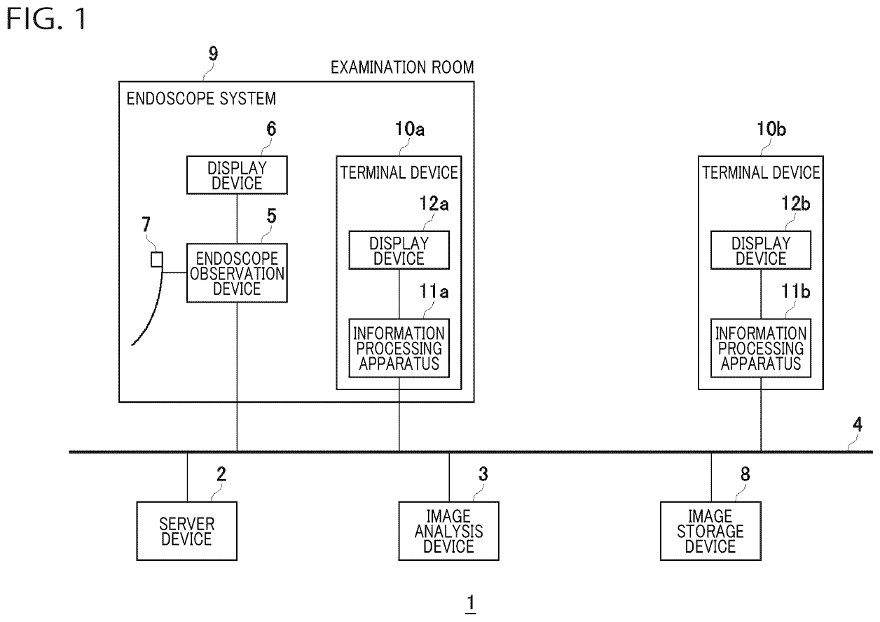

FIG. 1 shows the configuration of a medical support system 1 according to an embodiment. The medical support system 1 is provided in a medical facility such as a hospital where endoscopic examinations are performed. In the medical support system 1, a server device 2, an image analysis device 3, an image storage device 8, an endoscope system 9, and a terminal device 10b are communicably connected via a network 4 such as a local area network (LAN). The endoscope system 9 is installed in an examination room and has an endoscope observation device 5 and a terminal device 10a. In the medical support system 1, the server device 2, the image analysis device 3, and the image storage device 8 may be provided outside the medical facility, for example, in a cloud server.

The endoscope observation device 5 is connected to an endoscope 7 to be inserted into the digestive tract of a patient. The endoscope 7 has a light guide for illuminating the inside of the digestive tract by transmitting illumination light supplied from the endoscope observation device 5, and the distal end of the endoscope 7 is provided with an illumination window for emitting the illumination light transmitted by the light guide to living tissue and an image-capturing unit for image-capturing the living tissue at a predetermined cycle and outputting an image-capturing signal to the endoscope observation device 5. The image-capturing unit includes a solid-state imaging device, e.g., a CCD image sensor or a CMOS image sensor, that converts incident light into an electric signal.

The endoscope observation device 5 performs image processing on the image-capturing signal photoelectrically converted by a solid-state imaging device of the endoscope 7 so as to generate an endoscopic image and displays the endoscopic image on the display device 6 in real time. In addition to normal image processing such as A/D conversion and noise removal, the endoscope observation device 5 may include a function of performing special image processing for the purpose of highlighting, etc. The endoscope observation device 5 generates endoscopic images at a predetermined cycle, e.g., 1/60 seconds. The endoscope observation device 5 may be formed by one or more processors with dedicated hardware or may be formed by one or more processors with general-purpose hardware.

According to the examination procedure, the doctor observes an endoscopic image displayed on the display device 6. The doctor observes the endoscopic image while moving the endoscope 7, and operates the release switch of the endoscope 7 when a biological tissue to be captured appears on the display device 6. When the release switch is operated, the endoscope observation device 5 captures (saves) an endoscopic image at the time when the release switch is operated and then transmits the captured endoscopic image to the image storage device 8 along with information identifying the endoscopic image (image ID). The endoscope observation device 5 may transmit a plurality of captured endoscopic images all at once to the image storage device 8 after the examination is completed. The image storage device 8 records the endoscopic images transmitted from the endoscope observation device 5 in association with an examination ID for identifying the endoscopic examination.

In the embodiment, “imaging” refers to operation of converting incident light into an electrical signal performed by a solid-state image sensor of an endoscope 7, and “capturing” refers to operation of saving (recording) an endoscope image generated by the endoscope observation device 5. The “imaging” may include an operation of generating an endoscopic image from the converted electrical signal performed by the endoscope observation device 5.

The terminal device 10a is installed in the examination room with an information processing apparatus 11a and a display device 12a. The terminal device 10a may be used by doctors, nurses, and others in order to check information on a biological tissue being captured in real time during endoscopic examinations.

The terminal device 10b is installed in a room other than the examination room with an information processing apparatus 11b and a display device 12b. The terminal device 10b is used when a doctor creates a report of an endoscopic examination. The terminal devices 10a and 10b are formed by one or more processors having general-purpose hardware.

In the medical support system 1 according to the embodiment, the endoscope observation device 5 displays endoscopic images in real time through the display device 6, and provides the endoscopic images along with meta information of the images to the image analysis device 3 in real time. The meta information may be information that includes at least the frame number and imaging time information of each image, where the frame number indicates the number of the frame after the endoscope 7 starts imaging.

The image analysis device 3 is an electronic calculator (computer) that analyzes endoscopic images to detect lesions in the endoscopic images and performs qualitative diagnosis of the detected lesions. The image analysis device 3 may be a computer-aided diagnosis (CAD) system with an artificial intelligence (AI) diagnostic function. The image analysis device 3 may be formed by one or more processors with dedicated hardware or may be formed by one or more processors with general-purpose hardware.

The image analysis device 3 may use a trained model that is generated by machine learning using endoscopic images for learning and information concerning a lesion area contained in the endoscopic images as training data. Annotation work on the endoscopic images is performed by annotators with expertise, such as doctors, and machine learning may use CNN, RNN, LSTM, etc., which are types of deep learning. Upon input of an endoscopic image, this trained model outputs information indicating an imaged organ, information indicating an imaged site, and information concerning an imaged lesion (lesion information). The lesion information output by the image analysis device 3 includes at least information on the presence or absence of a lesion indicating whether the endoscopic image contains a lesion or not. When the lesion is contained, the lesion information may include information indicating the size of the lesion, information indicating the location of the outline of the lesion, information indicating the shape of the lesion, information indicating the invasion depth of the lesion, and a qualitative diagnosis result of the lesion. The qualitative diagnostic result of the lesion includes the type of lesion. During an endoscopic examination, the image analysis device 3 is provided with endoscopic images from the endoscope observation device 5 in real time and outputs information indicating the organ, information indicating the site, and lesion information for each endoscopic image.

The image analysis device 3 has a function of recognizing the image quality of an endoscopic image. The image analysis device 3 checks by image analysis whether blurring, jigging, clouding, or the like has occurred or residue is contained in the image, and determines whether or not the image has been properly captured. For each endoscopic image, the image analysis device 3 outputs image quality information indicating whether or not the image has been properly captured. Hereinafter, information indicating an organ, information indicating a site, lesion information, and image quality information that are output for each endoscopic image are collectively referred to as “image analysis information.”

When the user operates the release switch (capture operation), the endoscope observation device 5 provides the frame number, imaging time, and image ID of the captured endoscopic image to the image analysis device 3, along with information indicating that the capture operation has been performed (capture operation information). Upon acquiring the capture operation information, the image analysis device 3 provides the image ID, the frame number, the imaging time information, and image analysis information for the provided frame number to the server device 2, along with the examination ID. The image ID, the frame number, the imaging time information, and the image analysis information constitute “additional information” that expresses the features and properties of the endoscopic image. Upon acquiring the capture operation information, the image analysis device 3 transmits the additional information to the server device 2 along with the examination ID, and the server device 2 records the additional information in association with the examination ID.

When the user finishes the endoscopic examination, the user operates an examination completion button on the endoscope observation device 5. The operation information of the examination completion button is provided to the server device 2 and the image analysis device 3, and the server device 2 and the image analysis device 3 recognize the completion of the endoscopic examination. Upon receiving the examination completion information, the image analysis device 3 provides information indicating whether or not an endoscopic image including a lesion to be attached to the report has been detected in the endoscopic examination to the server device 2 along with the examination ID. Hereinafter, information indicating whether or not an endoscopic image including a lesion to be attached to the report has been detected is referred to as “lesion image existence information.” For example, when the image analysis device 3 detects even one endoscopic image including a lesion during the examination, the image analysis device 3 may provide the server device 2 with lesion image existence information indicating that the endoscopic image to be attached to the report has been detected. On the other hand, when the image analysis device 3 has not detected even one endoscopic image including a lesion during the examination, the image analysis device 3 may provide the server device 2 with lesion image existence information indicating that the endoscopic image to be attached to the report has not been detected.

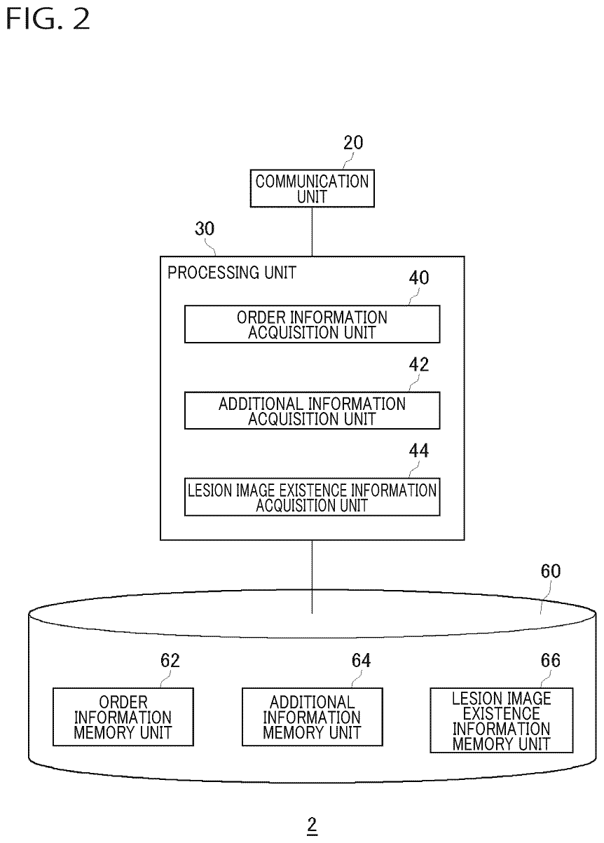

FIG. 2 shows functional blocks of the server device 2. The server device 2 includes a communication unit 20, a processing unit 30, and a memory device 60. The communication unit 20 transmits and receives information such as data and instructions between the image analysis device 3, the endoscope observation device 5, the image storage device 8, the terminal device 10a, and the terminal device 10b via the network 4. The processing unit 30 includes an order information acquisition unit 40, an additional information acquisition unit 42, and a lesion image existence information acquisition unit 44. The memory device 60 has an order information memory unit 62, an additional information memory unit 64, and a lesion image existence information memory unit 66.

The server device 2 includes a computer. Various functions shown in FIG. 2 are realized by the computer executing a program. The computer includes a memory for loading programs, one or more processors that execute loaded programs, auxiliary storage, and other LSIs as hardware. The processor may be formed with a plurality of electronic circuits including a semiconductor integrated circuit and an LSI, and the plurality of electronic circuits may be mounted on one chip or on a plurality of chips. The functional blocks shown in FIG. 2 are realized by cooperation between hardware and software. Therefore, a person skilled in the art should appreciate that there are many ways of accomplishing these functional blocks in various forms in accordance with the components of hardware only, software only, or the combination of both.

The order information acquisition unit 40 acquires order information for an endoscopic examination from a hospital information system. For example, before the start of the examination work for one day at the medical facility, the order information acquisition unit 40 acquires the order information for the day from the hospital information system and stores the order information in the order information memory unit 62. Before the start of the examination, the endoscope observation device 5 or the information processing apparatus 11a may read out order information for the examination to be performed from the order information memory unit 62 and display the order information on the display device.

The additional information acquisition unit 42 acquires the examination ID and additional information for the endoscopic image from the image analysis device 3, and stores the additional information in association with the examination ID in the additional information memory unit 64. The additional information for the endoscopic image includes an image ID, a frame number, imaging time information, and image analysis information. For example, if a user performs 17 capture operations during an endoscopic examination, the additional information acquisition unit 42 acquires additional information for 17 endoscopic images from the image analysis device 3 and stores the additional information in the additional information memory unit 64. Image IDs are assigned by the endoscope observation device 5, and the endoscope observation device 5 assigns the image IDs in order of the imaging time, starting from 1. Therefore, image IDs 1 to 17 are assigned to the 17 endoscopic images, respectively, in this case.

The lesion image existence information acquisition unit 44 acquires the examination ID and the lesion image existence information from the image analysis device 3, and stores the lesion image existence information in association with the examination ID in the lesion image existence information memory unit 66. The lesion image existence information acquisition unit 44 may acquire lesion image existence information from the image analysis device 3 after the examination is completed.

In the embodiment, when the image analysis device 3 has not detected a lesion in the endoscopic examination, the information processing apparatus 11b proposes a technique for automatically selecting an endoscopic image to be attached to the report of the examination. Therefore, in the embodiment, the lesion image existence information acquisition unit 44 acquires lesion image existence information indicating that no endoscopic image including the lesion to be attached to the report has been detected from the image analysis device 3 after the examination is completed.

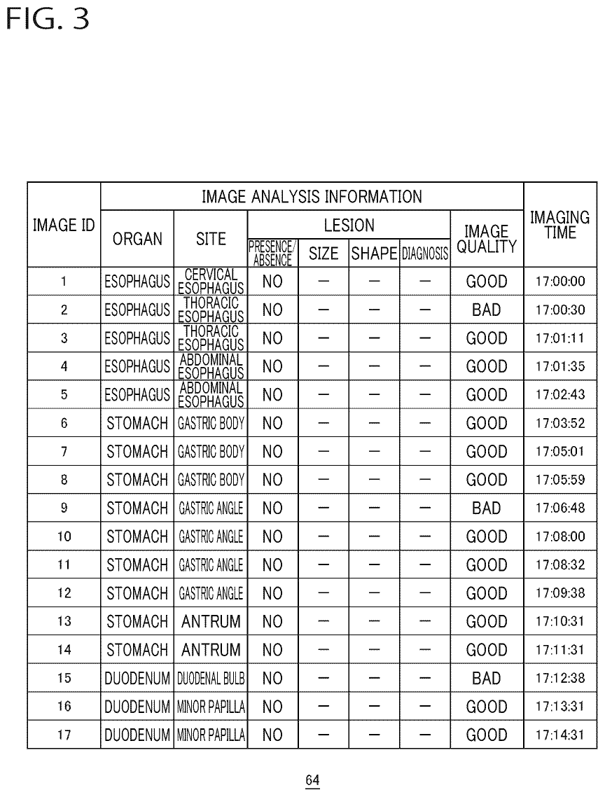

FIG. 3 shows examples of additional information linked to captured endoscopic images. An “ORGAN” field stores information indicating the organ included in the image, i.e., the organ that has been imaged, and a “SITE” field stores information indicating the site of the organ included in the image, i.e., the site of the organ that has been imaged.

A “PRESENCE/ABSENCE” field stores information indicating whether or not a lesion has been detected by the image analysis device 3 in the lesion information. As described above, in the embodiment, since the image analysis device 3 has not detected a lesion in the endoscopic examination, “NO” is stored in the “PRESENCE/ABSENCE” field of all the endoscopic images with the image IDs 1 to 17.

A “SIZE” field stores information indicating the longest diameter of the bottom surface of the lesion, a “SHAPE” field stores coordinate information expressing the contour shape of the lesion, and a “DIAGNOSIS” field stores the qualitative diagnosis result of the lesion. In the embodiment, no lesion is included in all the endoscopic images, and each field is therefore set to be blank.

In an “IMAGE QUALITY” field, information indicating whether or not the image has been properly captured is stored, “GOOD” indicates that the image has been properly captured, and “POOR” indicates that the image has not been properly captured. An “IMAGING TIME” field stores information indicating the imaging time of the image. The “IMAGING TIME” field may include a frame number.

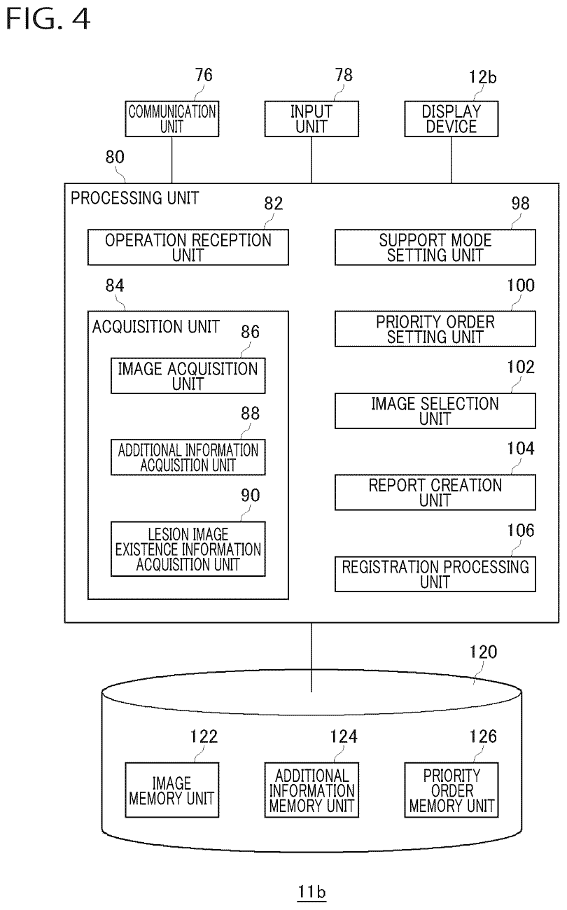

FIG. 4 shows the functional blocks of the information processing apparatus 11b. The information processing apparatus 11b has the function of automatically selecting endoscopic images to be attached to a report for an examination in which no lesion has been detected and includes a communication unit 76, an input unit 78, a processing unit 80, and a memory device 120. The communication unit 76 transmits and receives information such as data and instructions between the server device 2, the image analysis device 3, the endoscope observation device 5, the image storage device 8, and the terminal device 10a via the network 4. The processing unit 80 includes an operation reception unit 82, an acquisition unit 84, a support mode setting unit 98, a priority order setting unit 100, an image selection unit 102, a report creation unit 104, and a registration processing unit 106, and the acquisition unit 84 has an image acquisition unit 86, an additional information acquisition unit 88, and a lesion image existence information acquisition unit 90. The memory device 120 has an image memory unit 122, an additional information memory unit 124, and a priority order memory unit 126.

The information processing apparatus 11b includes a computer. Various functions shown in FIG. 4 are realized by the computer executing a program. The computer includes a memory for loading programs, one or more processors that execute loaded programs, auxiliary storage, and other LSIs as hardware. The processor may be formed with a plurality of electronic circuits including a semiconductor integrated circuit and an LSI, and the plurality of electronic circuits may be mounted on one chip or on a plurality of chips. The functional blocks shown in FIG. 4 are realized by cooperation between hardware and software. Therefore, a person skilled in the art should appreciate that there are many ways of accomplishing these functional blocks in various forms in accordance with the components of hardware only, software only, or the combination of both.

After the completion of an endoscopic examination, the user, a doctor, inputs a user ID and a password to the information processing apparatus 11b so as to log in. An application for preparing an examination report is activated when the user logs in, and a list of already performed examinations is displayed on the display device 12b. The list of already performed examinations displays examination information such as a patient name, a patient ID, examination date and time, an examination item, and the like in a list, and the user operates the input unit 78 such as a mouse or a keyboard so as to select an examination for which a report is to be created. When the operation reception unit 82 receives an examination selection operation, the image acquisition unit 86 acquires a plurality of endoscopic images linked to the examination ID of the examination selected by the user from the image storage device 8 and stores the endoscopic images in the image memory unit 122, and the additional information acquisition unit 88 acquires additional information linked to the examination ID of the examination selected by the user and stores the additional information in the additional information memory unit 124. The lesion image existence information acquisition unit 90 acquires the lesion image existence information linked to the examination ID of the examination selected by the user from the server device 2. The lesion image existence information is information indicating whether or not endoscopic images including a lesion to be attached to a report have been detected by the image analysis device 3 in the endoscopic examination.

The support mode setting unit 98 sets a mode in which the image selection unit 102 automatically selects an image to be attached to the report according to the lesion image existence information. When the image analysis device 3 has not detected a lesion in the endoscopic examination, the support mode setting unit 98 sets a first support mode in which the image selection unit 102 selects report attachment images based on the priority order. In the first support mode, the image selection unit 102 selects a predetermined amount of report attachment images from among a plurality of captured endoscopic images based on the priority order set for the additional information. The predetermined amount of report attachment images may mean a predetermined number of still images when the captured endoscope images are still images, and may mean moving images of a predetermined volume (or a predetermined time) when the captured endoscope images are moving images.

On the other hand, when the image analysis device 3 has detected a lesion in the endoscopic examination, the support mode setting unit 98 sets a second support mode in which the image selection unit 102 selects endoscopic images including the lesion. In the second support mode, the image selection unit 102 selects report attachment images including the lesion detected by the image analysis device 3 from among a plurality of captured endoscopic images.

In the following embodiment, an explanation will be given regarding the operation of the processing unit 80 when the lesion image existence information acquired by the lesion image existence information acquisition unit 90 indicates that the image analysis device 3 has not detected a lesion such that the support mode setting unit 98 sets the first support mode. During the examination, the user captures endoscopic images, which are still images.

In the first support mode, the image selection unit 102 automatically selects report attachment images based on the priority order set for the additional information. The priority order may be set for at least one of an organ, a site, and imaging timing. The priority order setting unit 100 sets the priority order based on input from the user and stores the priority order in the priority order memory unit 126. In the embodiment, “priority order” could also be phrased as “ranking”. For example, the user may select one priority from among a plurality of priority order candidates. By allowing the user to determine the criteria for automatically selecting report attachment images in an examination where lesions are undetected, a report reflecting the preferences of the user and the hospital facility can be created. The upper limit number (upper limit amount) of report attachment images may also be set by the user.

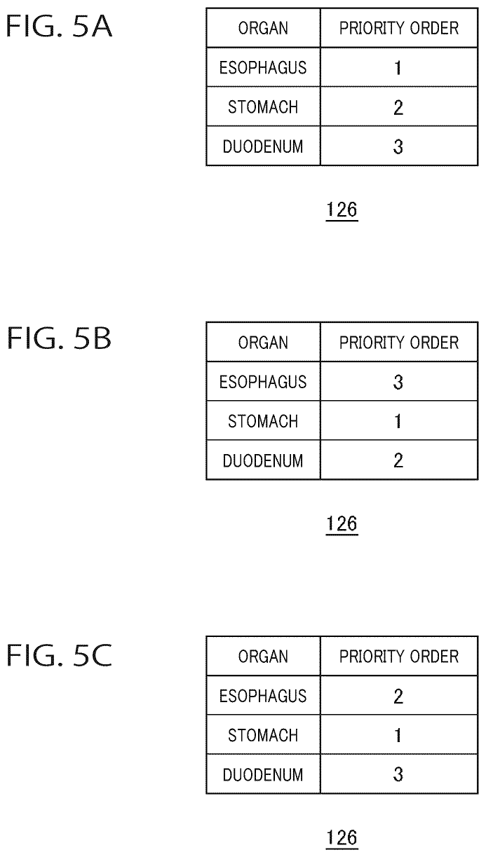

FIG. 5A is a diagram showing an example of priority order set for organs. In the priory order shown in FIG. 5A, the priority order of “esophagus” is set as the first priority, the priority order of “stomach” is set as the second priority, and the priority order of “duodenum” is set as the third priority. The image selection unit 102 selects report attachment images within the upper limit number of report attachment images from among a plurality of endoscopic images captured by the user according to priority order. The image selection unit 102 selects report attachment images one by one in order from the highest priority in reference to information indicating the organ included in the additional information of each endoscopic image. Once the image selection unit 102 selects a report attachment image with the lowest priority, the image selection unit 102 selects report attachment images in order from the highest priority again.

For example, when the upper limit number of the report attachment images is set to “5 images,” the image selection unit 102 selects a report attachment image including the esophagus as the first image, selects a report attachment image including the stomach as the second image, selects a report attachment image including the duodenum as the third image, selects a report attachment image including the esophagus as the fourth image, and selects a report attachment image including the stomach as the fifth image. As described above, the image selection unit 102 according to the embodiment selects report attachment images based on the set priority order within the upper limit number.

FIG. 5B is a diagram showing another example of priority order set for organs. In the priory order shown in FIG. 5B, the priority order of “stomach” is set as the first priority, the priority order of “duodenum” is set as the second priority, and the priority order of “esophagus” is set as the third priority. The priority order shown in FIG. 5B is set, and when the upper limit number of the report attachment images is set to “5 images,” the image selection unit 102 selects a report attachment image including the stomach as the first image, selects a report attachment image including the duodenum as the second image, selects a report attachment image including the esophagus as the third image, selects a report attachment image including the stomach as the fourth image, and selects a report attachment image including the duodenum as the fifth image.

FIG. 5C is a diagram showing another example of priority order set for organs. In the priory order shown in FIG. 5C, the priority order of “stomach” is set as the first priority, the priority order of “esophagus” is set as the second priority, and the priority order of “duodenum” is set as the third priority. The priority order shown in FIG. 5C is set, and when the upper limit number of the report attachment images is set to “5 images,” the image selection unit 102 selects a report attachment image including the stomach as the first image, selects a report attachment image including the esophagus as the second image, selects a report attachment image including the duodenum as the third image, selects a report attachment image including the stomach as the fourth image, and selects a report attachment image including the esophagus as the fifth image.

As described above, the priority order setting unit 100 sets the priority order based on input from the user. The image selection unit 102 selects report attachment images according to the priority order determined by the user, which allows a report creation unit 104 described later to create a report according to the user's requests. The priority order for organs shown in FIGS. 5A to 5C is an example, and the user may freely set the priory order for organs.

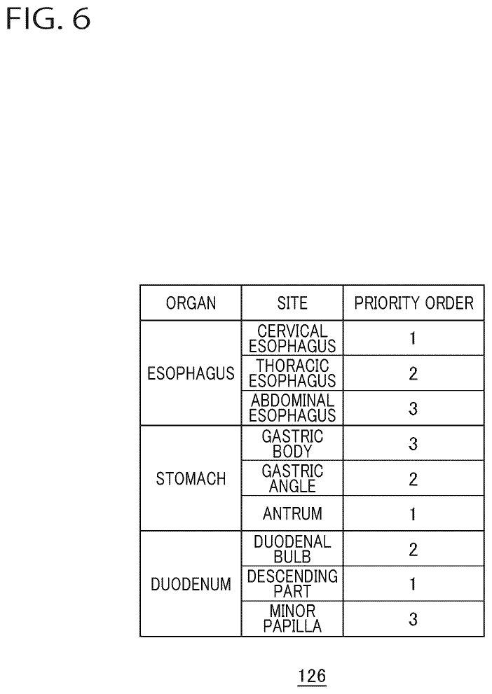

FIG. 6 shows an example of priority order set for sites of organs. This priority order sets the priority order of a site in each organ. According to the priority order shown in FIG. 6, the priority order of “cervical esophagus” is set to be the first priority, “thoracic esophagus” is set to be the second priority, “abdominal esophagus” is set to be the third priority in “esophagus”; “antrum” is set to be the first priority, “gastric angle” is set to be the second priority, and “gastric body” is set to be the third priority in “stomach”; and “descending part” is set to be the first priority, “duodenal bulb” is set to be the second priority, and “minor papilla” is set to be the third priority in “duodenum.” The priority order shown in FIG. 6 is an example, and the user may arbitrarily determine the priority order of a site in each organ.

The image selection unit 102 may select report attachment images based on the priority order set for the organs, e.g., the priority order shown in any one of FIGS. 5A to 5C, and the priority order set for the sites. Hereinafter, an explanation will be given regarding the operation of the image selection unit 102 of selecting report attachment images based on the priority order for the organs shown in FIG. 5A, hereinafter simply referred to as “organ priority order,” and the priority order for the sites shown in FIG. 6, hereinafter simply referred to as “site priority order.” The image selection unit 102 specifies endoscopic images including an organ specified based on the organ priority order shown in FIG. 5A, and when a plurality of endoscopic images including the organ have been acquired, the image selection unit 102 selects report attachment images from among the plurality of endoscopic images including the organ based on the site priority order shown in FIG. 6.

Based on the organ priority order shown in FIG. 5A and the site priority order shown in FIG. 6, the image selection unit 102 specifies the priority order of combinations of organs and sites for selecting the report attachment images.

### (Priority Order Identification Step 1)

Upon recognizing that a report attachment image to be selected for the first image is an image including “esophagus” based on the organ priority order shown in FIG. 5A, the image selection unit 102 recognizes that the priority order of “cervical esophagus” in “esophagus” is the first priority based on the site priority order shown in FIG. 6, and identifies that a combination of (esophagus, cervical esophagus) is the first priority.

### (Priority Order Identification Step 2)

Upon recognizing that a report attachment image to be selected for the second image is an image including “stomach” based on the organ priority order shown in FIG. 5A, the image selection unit 102 recognizes that the priority order of “antrum” in “stomach” is the first priority based on the site priority order shown in FIG. 6, and identifies that a combination of (stomach, antrum) is the second priority.

### (Priority Order Identification Step 3)

Upon recognizing that a report attachment image to be selected for the third image is an image including “duodenum” based on the organ priority order shown in FIG. 5A, the image selection unit 102 recognizes that the priority order of “descending part” in “duodenum” is the first priority based on the site priority order shown in FIG. 6, and identifies that a combination of (duodenum, descending part) is the third priority.

### (Priority Order Identification Step 4)

Upon recognizing that a report attachment image to be selected for the fourth image is an image including “esophagus” based on the organ priority order shown in FIG. 5A, the image selection unit 102 recognizes that the priority order of “thoracic esophagus” in “esophagus” is the second priority based on the site priority order shown in FIG. 6, and identifies that a combination of (esophagus, thoracic esophagus) is the fourth priority.

### (Priority Order Identification Step 5)

Upon recognizing that a report attachment image to be selected for the fifth image is an image including “stomach” based on the organ priority order shown in FIG. 5A, the image selection unit 102 recognizes that the priority order of “gastric angle” in “stomach” is the second priority based on the site priority order shown in FIG. 6, and identifies that a combination of (stomach, gastric angle) is the fifth priority.

### (Priority Order Identification Step 6)

Upon recognizing that a report attachment image to be selected for the sixth image is an image including “duodenum” based on the organ priority order shown in FIG. 5A, the image selection unit 102 recognizes that the priority order of “duodenal bulb” in “duodenum” is the second priority based on the site priority order shown in FIG. 6, and identifies that a combination of (duodenum, duodenal bulb) is the sixth priority.

### (Priority Order Identification Step 7)

Upon recognizing that a report attachment image to be selected for the seventh image is an image including “esophagus” based on the organ priority order shown in FIG. 5A, the image selection unit 102 recognizes that the priority order of “abdominal esophagus” in “esophagus” is the third priority based on the site priority order shown in FIG. 6, and identifies that a combination of (esophagus, abdominal esophagus) is the seventh priority.

### (Priority Order Identification Step 8)

Upon recognizing that a report attachment image to be selected for the eighth image is an image including “stomach” based on the organ priority order shown in FIG. 5A, the image selection unit 102 recognizes that the priority order of “gastric body” in “stomach” is the third priority based on the site priority order shown in FIG. 6, and identifies that a combination of (stomach, gastric body) is the eighth priority.

### (Priority Order Identification Step 9)

Upon recognizing that a report attachment image to be selected for the ninth image is an image including “duodenum” based on the organ priority order shown in FIG. 5A, the image selection unit 102 recognizes that the priority order of “minor papilla” in “duodenum” is the third priority based on the site priority order shown in FIG. 6, and identifies that a combination of (duodenum, minor papilla) is the ninth priority.

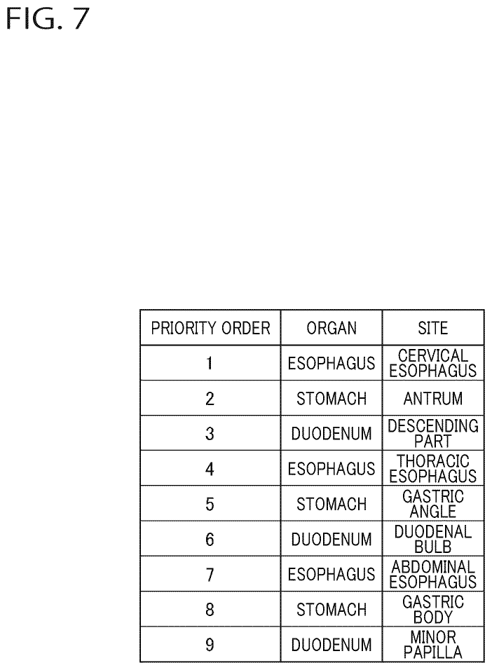

FIG. 7 shows priority order specified based on the organ priority order shown in FIG. 5A and the site priority order shown in FIG. 6. When the upper limit number is five, the image selection unit 102 selects a report attachment image including the cervical esophagus of the esophagus as the first image, selects a report attachment image including the antrum of the stomach as the second image, selects a report attachment image including the descending part of the duodenum as the third image, selects a report attachment image including the thoracic esophagus of the esophagus as the fourth image, and selects a report attachment image including the gastric angle of the stomach as the fifth image, according to the priority order shown in FIG. 7. As described above, the image selection unit 102 may select report attachment images based on the priority order derived for the organs and the sites within the upper limit number.

When there is no endoscopic image including a combination of (organ, site) in the additional information, the image selection unit 102 selects a report attachment image including a site with a lower priority in the same organ. For example, if there is no endoscopic image including the descending part of the duodenum when the image selection unit 102 attempts to select a report attachment image including the descending part of the duodenum, the image selection unit 102 selects a report attachment image including the duodenal bulb of the duodenum. If there is no endoscopic image including the duodenal bulb of the duodenum, the image selection unit 102 selects a report attachment image including the minor papilla of the duodenum. In this way, by selecting report attachment images based on the organ priority order, the image selection unit 102 can select report attachment images of each organ evenly.

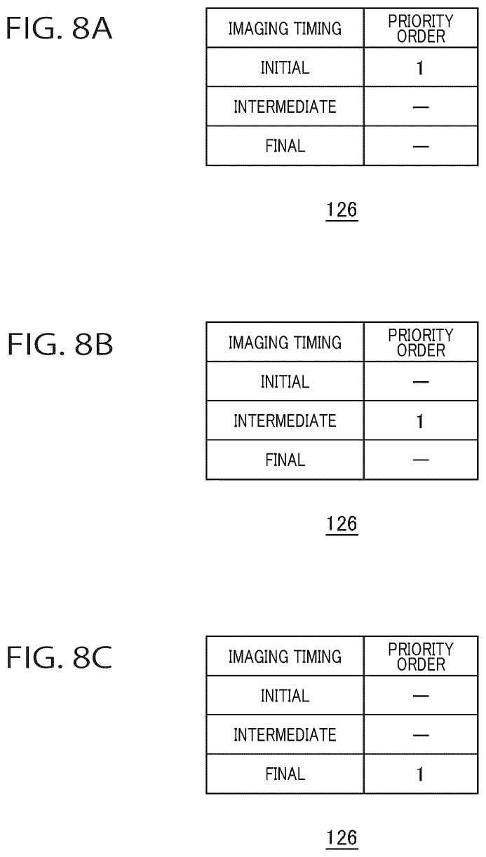

FIG. 8A shows an example of priority order set for the order of imaging. When a plurality of endoscopic images including the same organ or the same site have been acquired, the priority order set for the order of imaging is used to select one report attachment image from among the plurality of endoscopic images including the organ or the site.

As the priority order for the order of imaging, the priority order setting unit 100 sets any one of a first priority criterion for preferentially selecting an endoscopic image captured at an initial point in time, a second priority criterion for preferentially selecting an endoscopic image captured at an intermediate point in time, and a third priority criterion for preferentially selecting an endoscopic image captured at a final point in time. In the example shown in FIG. 8A, the first priority criterion is set.

For example, when the organ priority order shown in FIG. 5A and the priority order in the order of imaging shown in FIG. 8A are set as the priority order for image selection, the image selection unit 102 refers to information indicating the imaging time included in the additional information of endoscopic images including the esophagus and selects the earliest-captured endoscopic image including the esophagus as a first report attachment image.

FIG. 8B shows another example of priority order set for the order of imaging. In the example shown in FIG. 8B, the second priority criterion is set. When the second priority criterion is set, the image selection unit 102 selects an endoscopic image captured at an intermediate point in time in the imaging order from among the plurality of endoscopic images as a report attachment image.

FIG. 8C shows another example of priority order set for the order of imaging. In the example shown in FIG. 8C, the third priority criterion is set. When the third priority criterion is set, the image selection unit 102 selects the last-captured endoscopic image from among the plurality of endoscopic images as a report attachment image. In this way, the image selection unit 102 can narrow down the number of report attachment images to one by taking into account the priority order in the order of imaging.

Hereinafter, an explanation will be given regarding a procedure for the image selection unit 102 to select report attachment images based on the organ priority order shown in FIG. 5A, the site priority order shown in FIG. 6, and the priority order in the order of imaging shown in FIG. 8C. The upper limit number of report attachment images is set to “five.” The endoscopic images acquired during the examination are 17 images, and the additional information acquisition unit 88 acquires additional information of the endoscope images shown in FIG. 3 and stores the additional information in the additional information memory unit 124.

### (Procedure for Selecting First Report Attachment Image)

When the image selection unit 102 specifies that the combination of (esophagus, cervical esophagus) is the first priority based on the organ priority order and the site priority order, the image selection unit 102 refers to the additional information of each endoscopic image and searches for endoscopic images having image analysis information of (esophagus, cervical esophagus). In reference to FIG. 3, an endoscope image with an image ID 1 meets this requirement. Therefore, the image selection unit 102 selects the endoscopic image with the image ID 1 as the first report attachment image.

### (Procedure for Selecting Second Report Attachment Image)

When the image selection unit 102 specifies that the combination of (stomach, antrum) is the second priority based on the organ priority order and the site priority order, the image selection unit 102 refers to the additional information of each endoscopic image and searches for endoscopic images having image analysis information of (stomach, antrum). In reference to FIG. 3, two endoscopic images with image IDs 13 and 14 meet this requirement. Since the priority order in the order of imaging shown in FIG. 8C is set to the third priority criterion for preferentially selecting an endoscopic image captured at a final point in time, the image selection unit 102 selects an endoscopic image with the image ID 14 captured late as the second report attachment image.

### (Procedure for Selecting Third Report Attachment Image)

When the image selection unit 102 specifies that the combination of (duodenum, descending part) is the third priority based on the organ priority order and the site priority order, the image selection unit 102 refers to the additional information of each endoscopic image and searches for endoscopic images having image analysis information of (duodenum, descending part). In reference to FIG. 3, since there is no endoscopic image having image analysis information of (duodenum, descending part), the image selection unit 102 searches for endoscopic images having image analysis information of “duodenal bulb” whose site priority order is low by one in the duodenum. In FIG. 3, the endoscopic image with the image ID 15 meets this requirement.

When the image selection unit 102 refers to the “IMAGE QUALITY” field linked to the image ID 15 and recognizes that “BAD” indicating that the image has not been properly captured is stored, the image selection unit 102 determines not to select the endoscopic image with the image ID 15 as a report attachment image. Therefore, the image selection unit 102 searches for endoscopic images having image analysis information of “minor papilla” whose site priority order is low by one in the duodenum. In reference to FIG. 3, two endoscopic images with image IDs 16 and 17 meet this requirement. Since the priority order in the order of imaging shown in FIG. 8C is set to the third priority criterion, the image selection unit 102 selects a late-captured endoscopic image with the image ID 17 as the third report attachment image.

### (Procedure for Selecting Fourth Report Attachment Image)

When the image selection unit 102 specifies that the combination of (esophagus, thoracic esophagus) is the fourth priority based on the organ priority order and the site priority order, the image selection unit 102 refers to the additional information of each endoscopic image and searches for endoscopic images having image analysis information of (esophagus, thoracic esophagus). In reference to FIG. 3, two endoscopic images with the image IDs 2 and 3 meet this requirement.

The image selection unit 102 refers to the “IMAGE QUALITY” fields linked to the image IDs 2 and 3, and recognizes that “BAD” is stored in the “IMAGE QUALITY” field for the image ID 2. Therefore, the image selection unit 102 determines not to select the endoscopic image with the image ID 2 as a report attachment image, and selects the endoscopic image with the image ID 3 as the fourth report attachment image.

### (Procedure for Selecting Fifth Report Attachment Image)

When the image selection unit 102 specifies that the combination of (stomach, gastric angle) is the fifth priority based on the organ priority order and the site priority order, the image selection unit 102 refers to the additional information of each endoscopic image and searches for endoscopic images having image analysis information of (stomach, gastric angle). In reference to FIG. 3, four endoscopic images with the image IDs 9, 10, 11 and 12 meet this requirement.

When the image selection unit 102 refers to the “IMAGE QUALITY” fields linked to the image IDs 9, 10, 11 and 12 and recognizes that “BAD” is stored in the “IMAGE QUALITY” field for the image ID 9, the image selection unit 102 determines not to select the endoscopic image with the image ID 9 as a report attachment image. Subsequently, the image selection unit 102 selects the last-captured endoscopic image with the image ID 12 as the fifth report attachment image based on the priority order in the order of imaging shown in FIG. 8C.

According to the above selection procedure, the image selection unit 102 selects five report attachment images, which is the upper number. By setting the priority order in advance by the user, endoscopic images that reflect the user's preferences are automatically attached to the report.

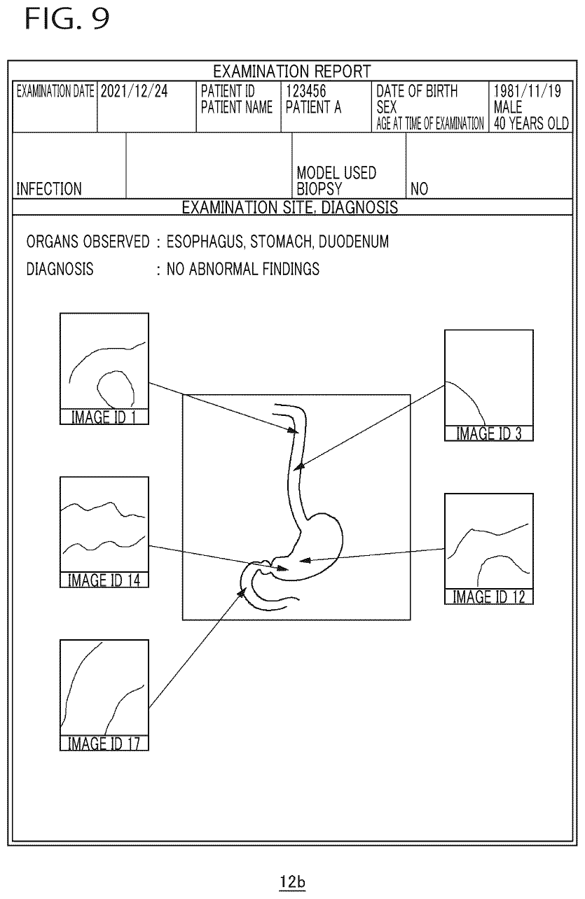

FIG. 9 shows an example of a report that is automatically created. When the support mode setting unit 98 sets the first support mode, the image selection unit 102 selects report attachment images based on the priority order set for the additional information as described above, and the report creation unit 104 automatically creates a report using the selected report attachment images.

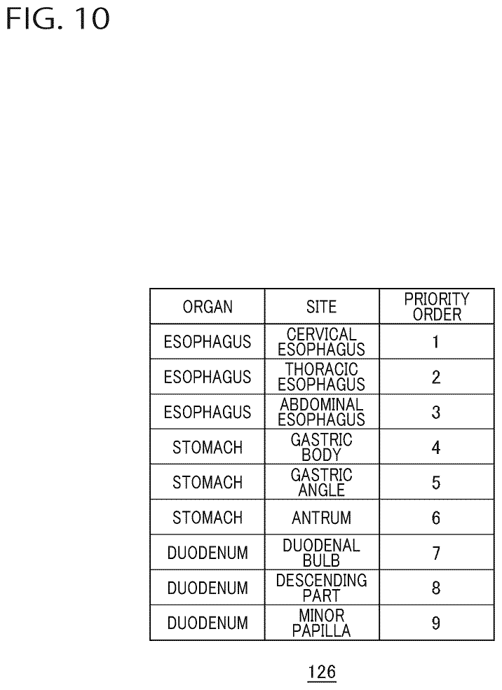

FIG. 10 shows an example of an automatically created report. The report creation unit 104 creates a report including information indicating that there is no lesion and report attachment images selected by the image selection unit 102, and displays the report on the display device 12b. When the image selection unit 102 selects a report attachment image, the report creation unit 104 may create a report that includes the details of the examination order, diagnosis indicating that there are no abnormal findings, and the report attachment image. The user may check the report displayed on the display device 12b and operate a predetermined registration button (not shown) so that the registration processing unit 106 registers the automatically created report in the server device 2.

Described above is an explanation based on the embodiments of the present disclosure. The embodiments are intended to be illustrative only, and it will be obvious to those skilled in the art that various modifications to constituting elements and processes could be developed and that such modifications are also within the scope of the present disclosure.

FIG. 10 shows an exemplary variation of the priority order set for combinations of organs and sites. In the embodiment, priority order is set according to combinations of organ priority order shown in FIGS. 5A to 5C and the site priority order shown in FIGS. 6A to 6B; however, the priority order shown in FIG. 10 is determines for combinations of the organs and the sites. Hereinafter, an explanation will be given regarding a procedure for the image selection unit 102 to select report attachment images based on the priority order shown in FIG. 10 and the priority order in the order of imaging shown in FIG. 8C. The upper limit number of report attachment images is set to “five.” The additional information acquisition unit 88 acquires additional information of the endoscope images shown in FIG. 3 and stores the additional information in the additional information memory unit 124.

### (Procedure for Selecting First Report Attachment Image)

When the image selection unit 102 recognizes that the combination of (esophagus, cervical esophagus) is the first priority, the image selection unit 102 refers to the additional information of each endoscopic image and searches for endoscopic images having image analysis information of (esophagus, cervical esophagus). In reference to FIG. 3, an endoscope image with an image ID 1 meets this requirement. Therefore, the image selection unit 102 selects the endoscopic image with the image ID 1 as the first report attachment image.

### (Procedure for Selecting Second Report Attachment Image)

When the image selection unit 102 recognizes that the combination of (esophagus, thoracic esophagus) is the second priority, the image selection unit 102 refers to the additional information of each endoscopic image and searches for endoscopic images having image analysis information of (esophagus, thoracic esophagus). In reference to FIG. 3, two endoscopic images with the image IDs 2 and 3 meet this requirement.

The image selection unit 102 refers to the “IMAGE QUALITY” fields linked to the image IDs 2 and 3, and recognizes that “BAD” is stored in the “IMAGE QUALITY” field for the image ID 2. Therefore, the image selection unit 102 determines not to select the endoscopic image with the image ID 2 as a report attachment image, and selects the endoscopic image with the image ID 3 as the second report attachment image.

### (Procedure for Selecting Third Report Attachment Image)

When the image selection unit 102 recognizes that the combination of (esophagus, abdominal esophagus) is the third priority, the image selection unit 102 refers to the additional information of each endoscopic image and searches for endoscopic images having image analysis information of (esophagus, abdominal esophagus). In reference to FIG. 3, two endoscopic images with the image IDs 4 and 5 meet this requirement. Since the priority order in the order of imaging shown in FIG. 8C is set to the third priority criterion for preferentially selecting an endoscopic image captured at a final point in time, the image selection unit 102 selects an endoscopic image with the image ID 5 captured late as the third report attachment image.

### (Procedure for Selecting Fourth Report Attachment Image)

When the image selection unit 102 recognizes that the combination of (stomach, gastric body) is the fourth priority, the image selection unit 102 refers to the additional information of each endoscopic image and searches for endoscopic images having image analysis information of (stomach, gastric body). In reference to FIG. 3, three endoscopic images with the image IDs 6, 7, and 8 meet this requirement. Since the priority order in the order of imaging shown in FIG. 8C is set to the third priority criterion, the image selection unit 102 selects the last-captured endoscopic image with the image ID 8 as the fourth report attachment image.

### (Procedure for Selecting Fifth Report Attachment Image)

When the image selection unit 102 specifies that the combination of (stomach, gastric angle) is the fifth priority, the image selection unit 102 refers to the additional information of each endoscopic image and searches for endoscopic images having image analysis information of (stomach, gastric angle). In reference to FIG. 3, four endoscopic images with the image IDs 9, 10, 11 and 12 meet this requirement.

When the image selection unit 102 refers to the “IMAGE QUALITY” fields linked to the image IDs 9, 10, 11 and 12 and recognizes that “BAD” is stored in the “IMAGE QUALITY” field for the image ID 9, the image selection unit 102 determines not to select the endoscopic image with the image ID 9 as a report attachment image. Subsequently, the image selection unit 102 selects the last-captured endoscopic image with the image ID 12 as the fifth report attachment image based on the priority order in the order of imaging shown in FIG. 8C.

According to the above selection procedure, the image selection unit 102 selects five report attachment images, which is the upper number. By setting the priority order for all the combinations of the organs and the sites in advance by the user, endoscopic images that reflect the user's preferences are automatically attached to the report.

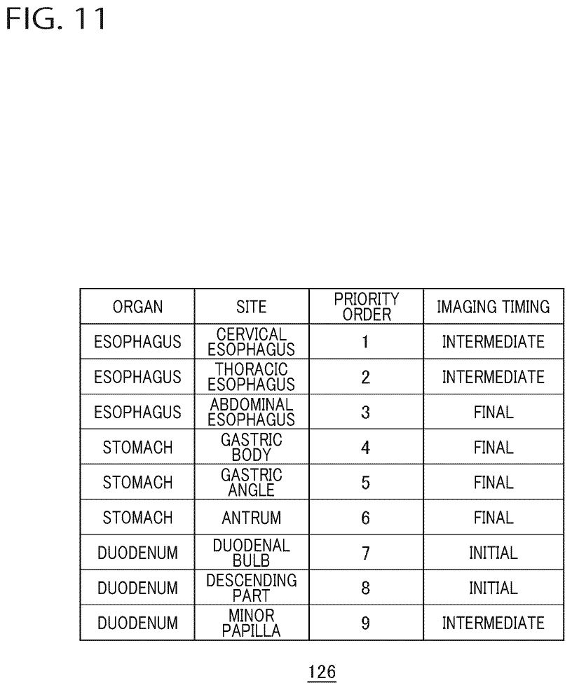

FIG. 11 shows an exemplary variation of the priority order set for combinations of organs and sites. In this example, the combinations of the organs and the sites are further associated with the priority order in imaging time. Setting the priority order in the imaging time for the combinations of the organs and the sites as described allows for the selection of report attachment images that reflect the user's preference in more detail.

In the embodiment, it has been explained that the user is able to set the upper limit number (upper limit amount) of report attachment images. The user may be able to select the upper limit number starting from zero. When the user sets the upper limit number of the images to zero such that the support mode setting unit 98 sets the first support mode, the image selection unit 102 operates so as not to automatically select report attachment images.

In the embodiment, a case where an endoscopic examination is an upper endoscopy has been explained; however, the endoscopic examination may be a lower endoscopy. The priority order setting unit 100 may set priority order for each examination item based on input from the user. For example, the priority order setting unit 100 may set the priority order for imaging time to be the third priority criterion for upper endoscopy reports, and set the priority order for imaging time to be the second priority criterion for lower endoscopy reports. In this case, the image selection unit 102 selects report attachment images for the upper endoscopy based on the third priority criterion while selecting report attachment images for the lower endoscopy based on the second priority criterion. Since the organs to be examined in the lower endoscopy are long, images captured at an intermediate point in time in the lower endoscopy are preferred as report attachment images representing the condition of the organs.

In the embodiment, the endoscope observation device 5 transmits captured images to the image storage device 8. However, in an exemplary variation, the image analysis device 3 may transmit captured images to the image storage device 8. In the embodiment, the information processing apparatus 11b has the processing unit 80. However, in the exemplary variation, the server device 2 may have the processing unit 80.

What is claimed is:

1. A medical support system comprising:
one or more processors comprising hardware, wherein the one or more processors are configured to:
acquire a plurality of endoscopic images captured in an endoscopic examination;
acquire respective pieces of additional information of the plurality of endoscopic images; and
select a predetermined amount of report attachment images from among the plurality of endoscopic images based on priority order set for the pieces of additional information when no endoscopic image including a lesion that is to be attached to a report is detected by a computer in the endoscopic examination.

2. The medical support system according to claim 1, wherein
the one or more processors are configured to:
create a report including information indicating that there is no lesion, and the report attachment images.

3. The medical support system according to claim 1, wherein
the one or more processors are configured to:
select the report attachment images based on the priority order when no endoscopic image including a lesion that is to be attached to the report is detected by the computer in the endoscopic examination; and
select the report attachment images including the lesion from among the plurality of endoscopic images when an endoscopic image including the lesion that is to be attached to the report is detected by the computer in the endoscopic examination.

4. The medical support system according to claim 1, wherein
the one or more processors are configured to:
set the priority order based on input from a user.

5. The medical support system according to claim 4, wherein
the one or more processors are configured to:
set the priority order for each examination item based on input from a user.

6. The medical support system according to claim 1, wherein
the additional information includes information indicating an organ included in the endoscopic images, and the one or more processors are configured to:
select the report attachment images based on priority order set for the organ.

7. The medical support system according to claim 6, wherein
the additional information includes information indicating a site of the organ included in the endoscopic images, and
the one or more processors are configured to:
select the report attachment images based on priority order set for the site of the organ.

8. The medical support system according to claim 1, wherein
the additional information includes information indicating order of imaging of the endoscopic images, and
the one or more processors are configured to:
when a plurality of endoscopic images including the same organ or the same site have been acquired, select the report attachment images from among the plurality of endoscopic images including the organ or the site based on the priority order set for the order of imaging.

9. The medical support system according to claim 8, wherein
the one or more processors are configured to:
as the priority order for the order of imaging, set any one of a first priority criterion for preferentially selecting the endoscopic image captured at an initial point in time, a second priority criterion for preferentially selecting the endoscopic image captured at an intermediate point in time, and a third priority criterion for preferentially selecting the endoscopic image captured at a final point in time.

10. The medical support system according to claim 7, wherein
the one or more processors are configured to:
when the plurality of endoscopic images including the same organ have been acquired, select the report attachment images from among the plurality of endoscopic images including the organ based on the priority order set for a site of the organ.

11. The medical support system according to claim 7, wherein
the one or more processors are configured to:
select the report attachment images based on priority order set for a combination of an organ and a site.

12. The medical support system according to claim 1, wherein
the one or more processors are configured to:
set the upper limit amount of the report attachment images based on input from a user; and
the user is able to select the upper limit amount starting from zero.

13. The medical support system according to claim 9, wherein
the endoscopic examination includes upper endoscopy and lower endoscopy,
the one or more processors are configured to:
select the report attachment images for upper endoscopy based on the third priority criterion, and
select the report attachment images for lower endoscopy based on the second priority criterion.

14. The medical support system according to claim 1, wherein
the one or more processors are configured to:
as the report attachment images, not select the endoscopic images in which the additional information includes information indicating that the images have not been properly captured.

15. A report creation support method comprising:
acquiring a plurality of endoscopic images captured in an endoscopic examination;
acquiring respective pieces of additional information of the plurality of endoscopic images; and
selecting a predetermined amount of report attachment images from among the plurality of endoscopic images based on priority order set for the pieces of additional information when no endoscopic image including a lesion that is to be attached to a report is detected by a computer in the endoscopic examination.

16. The report creation support method according to claim 15, comprising:
setting the priority order based on input from a user.

17. An information processing apparatus comprising: one or more processors comprising hardware, wherein
the one or more processors are configured to:
acquire a plurality of endoscopic images captured in an endoscopic examination;
acquire respective pieces of additional information of the plurality of endoscopic images; and
select a predetermined amount of report attachment images from among the plurality of endoscopic images based on priority order set for the pieces of additional information when no endoscopic image including a lesion that is to be attached to a report is detected by a computer in the endoscopic examination.

18. The information processing apparatus according to claim 17, wherein
the one or more processors are configured to:
set the priority order based on input from a user.
